(19) United States
(12) Patent Application Publication
(10) Pub. No.: US 2012/0266753 A1
(43) Pub. Date: Oct. 25, 2012

(54) HYGIENIC BEDDING INCLUDING MICROBIAL DETECTION VALVES AND INFECTION CONTROLS CUSHIONING AND/OR BEDDING DEVICES COMPRISING SAID MICROBIAL DETECTION VALVES

(75) Inventors: David Woolfson, Sandycove (IE); William Navan, Howth (IE); Duncan Bain, Kings Langley (GB)

(73) Assignee: Pneuma Pure I.P. Limited, Dublin (IE)

(21) Appl. No.: 13/510,566

(22) PCT Filed: Nov. 18, 2010
(86) PCT No.: PCT/EP2010/067780

§ 371 Date: Jun. 18, 2012

(30) Foreign Application Priority Data
Nov. 18, 2009 (IE) S2009/0876
Nov. 18, 2009 (IE) S2009/0877

(51) Int. Cl. B01D 53/22; C12M 1/34

(52) U.S. Cl. 96/6; 435/287.1

(57) ABSTRACT

The present invention relates to microbial detection valves and particularly, unidirectional valves which include means for detecting the presence of microbes. The present invention also relates to infection control cushioning and/or bedding including said microbial detection valves. In another aspect, the present invention concerns improvements in and relating to hygienic bedding and particularly in and relating to hygienic bedding covers. The present invention has the advantage that it provides a one-way valve incorporated into the pillow cover as well as a microbial filter membrane, to allow air to exhaust quickly from the pillow through the valve, bypassing the membrane. Air re-entering the pillow may only pass through the microbial filter membrane. Thus, air re-entering the pillow does so at slower speed, but has the advantage that forcing the re-entering air to pass through the micorbial filter allows only sterile air to enter the pillow. The present invention also provides a means for sealing hygienic bedding such as a mattress in a waterproof covering while enabling visual inspection.

The present invention concerns improvements in and relating to hygienic bedding and particularly in and relating to hygienic bedding covers. In another aspect, the present invention relates to microbial detection valves and particularly, unidirectional valves which include means for detecting the presence of microbes. The present invention also relates to infection control cushioning and/or bedding devices including these microbial detection valves.

Throughout this specification, it is to be understood that the term “hygienic bedding” includes infection control cushioning and/or bedding devices/products.

Known hygienic bedding, including mattresses, pillows, duvets, seat cushions, and postural supports such as gutter-splints, particularly for use in hospitals, are widely recognised as reservoirs for infection. Typically filled with polymer foam or other porous, spongy materials, such hygienic bedding provides an ideal harbour for breeding microbial organisms. This problem is greatly exacerbated in the presence of moisture.

Cross-infection is known to take place in hospitals by means of microbial (such as bacterial) ingress and subsequent incubation in the bedding, and subsequent escape of disease-causing microbes into the environment.

For this reason, in recent decades, hospital bedding has been encapsulated in waterproof materials. Many of these materials are permeable to water vapour, but not to liquid water. A typical example of such waterproof material is knitted nylon coated with polyurethane.

However, encapsulation of hygienic bedding such as a pillow for example, creates a new problem, namely that for the spongy materials within the pillow to yield to the shape of the head/body, when weight bears down on the pillow, air must be exhausted from the pillow/cushioning device, and when the weight is removed, air must be allowed to return to the pillow/cushioing device.

A partial solution to this problem is disclosed in Irish Patent No. 585166 and International Patent Publication No. WO 2009/034193 (Patent Application No. PCT/EP2008/062265) which disclose a non-woven PTFE membrane coated medium or other suitable filter provided as a patch forming part of the pillow cover. This filter material allowed the transport of air across the filter but had sufficiently small pore sizes in the PTFE membrane coated medium to prevent the transport of microbes, microbial spores, or of aerosol water droplets containing microbes.

However, the remaining disadvantage with use of this filter membrane is that air is not allowed to flow at an adequate rate to allow quick collapse of the pillow when weight is applied when laying the head down. This creates a peculiar sensation for the user giving the impression that he/she is sinking for a few seconds while the air seeps, under pressure, out through the filter membrane.

In another aspect, the present invention relates to microbial detection valves and particularly, unidirectional valves which include means for detecting the presence of microbes. In a further aspect, the present invention also relates to infection control cushioning and/or bedding devices including said microbial detection valves.

PCT Patent Publication No. WO 2009/034193 (Application No. PCT/EP2008/062265) discloses the inclusion of air-permeable waterproof microbial filter media, to allow air to pass in and out of the bedding without carrying air-borne microbes or spores.

In the case of certain forms of hygienic bedding, particularly, mattresses and cushions, it is often deemed desireable to allow inspection of the interior of the bedding. This is typically done in hospitals to check that the interior of a mattress has not become contaminated. Contamination of the mattress interior may have happened in spite of the use of waterproof encapsulation as described above, as a result of puncture, delamination of the waterproof coating to the cover fabric, or by other means. To allow inspection, a zip or other closure is typically provided in the waterproof coating.

In the case of the present invention, use of a conventional zip in the waterproof coating would compromise the functionality of a mattress including microbial filter media for filtering air since unfiltered air could enter the mattress at any time through the closed zip.

One way to overcome this could be by using a waterproof, air-tight zip. However, such a method of closure is prohibitively expensive.

In a further aspect of the infection control, hygienic bedding of the present invention, in the case of mattresses and cushions, it is often deemed desirable to enable inspection of the interior of the mattress. Inspection of the interior of the mattress is typically done in hospitals to check that the interior has not become contaminated. This may have happened in spite of the measures above, for instance as a result of puncture, delamination of the waterproof coating to the cover fabric, or by other means. To allow inspection, a zip or other closure is typically provided in the waterproof covering.

In the case of the present invention, a conventional zip would compromise the functionality of the mattress as a sterile area, since unfiltered air could enter the mattress at any time through the closed zip.

One way to overcome this is by using a waterproof, air-tight zip. However, such a method of closure is prohibitively expensive.

The present invention seeks to alleviate the above disadvantages associated with known hygienic bedding.

The present invention provides a valve for attachment to the cover of an infection control cushioning and/or bedding device e.g. a pillow and/or mattress cover and such like, the valve including means for detecting presence of microbes in the air flowing through the valve, thereby making it possible to identify the presence of pathogens, or metabolic products of pathogens, contained in the air passing through the valve.

Preferably, the valve is uni-directional, i.e. allows air to flow in one direction only; preferably wherein the valve only allows air to flow outwardly from an infection control cushioning and/or bedding device in which the valve is included.

Optionally, the microbial detection means includes at least one of the following group: a microbial detection reagent; a pH-sensitive colourant for indicating presence of microbes; metal complexes and solvatochromatic colourants.

Preferably, the valve comprises oil and the microbial detection means is included in the oil.

In another aspect, the present invention provides hermetically sealed infection control cushioning and/or bedding device such as a mattress, cushion, pillow, duvet, or such like having a sealed cover and having a resiliently deformable filing material comprising a filter medium including a filter membrane for the removal of particles of microbial size whereby the filter medium allows air and water vapour to flow through but is substantially impermeable to liquid while providing a barrier to microbial sized particles and wherein the infection control cushioning and/or bedding device also includes at least one valve which has microbial detection means associated with said valve.

Preferably, the valve is uni-directional, i.e. allows air to flow in one direction only, wherein the valve only allows air to flow outwardly from the hermetically sealed infection control cushioning and/or bedding device.

In one embodiment, the valve includes the microbial detection means.

Optionally, the microbial detection means includes at least one of the following group: a microbial detection reagent; a pH-sensitive colourant for indicating presence of microbes; metal complexes and solvatochromatic colourants.

Advantageously, the valve may comprise oil and the microbial detection means is included in the oil.

In an alternative embodiment, the microbial detection means associated with the valve comprises a two-part detection means whereby:
a first component of the detection means is contained within the hermetically sealed infection control cushioning and/or bedding device; the first component comprises an activator material which is releasable or degradable as a consequence of degradation; the valve includes a second component of the detection means, the second component comprising an indicator which is specific to detecting the activator material included in the first component of the detection means, whereby the indicator substance detects the presence of the activator material released from the first compoment of the detection means which, in turn, indicates microbial presence within the hermetically sealed infection control cushion and bedding device.

Advantageously, the activator material may be contained within a degradable carrier in the first component of the detection means, whereby degradation of the carrier results in release of the activator material; optionally, the activator material is encapsulated in the degradable carrier.

Preferably, the degradation is due to bacterialogical or other microbial presence in the cushioning and/or bedding device.

In a further aspect, the present invention provides hermetically sealed infection control cushioning and/or bedding device such as a mattress, cushion, pillow, duvet, or such like having a sealed cover and having a resiliently deformable filling material comprising a filter medium including a filter membrane for the removal of particles of microbial size whereby the filter medium allows air and water vapour flow through but is substantially impermeable to liquid while providing a barrier to microbial sized particles and wherein the infection control bedding product also includes a uni-directional valve to allow air to be vented from the pillow whereby any air entering the pillow is forced to pass through the filter.

Preferably, one part of the cover includes an area comprising a waterproof microbial filter membrane, and elsewhere on the cover is attached a unidirectional exhaust valve; optionally wherein the valve may be incorporated into the filter area of the cover in the area comprising the waterproof microbial filter membrane.

In a further aspect, the present invention provides a hermetically sealed infection control cushioning and/or bedding device such as a mattress, cushion, pillow, duvet, or such like having a sealed cover and having a resiliently deformable filling material comprising a filter medium in the cover, wherein the filter medium comprises a filter membrane for the removal of particles of microbial size whereby the filter medium allows air and water vapour flow through but is substantial impermeable to liquid while providing a barrier to microbial sized particles and wherein the infection control bedding product also includes a uni-directional valve to allow air to be vented from the pillow whereby any air entering the pillow is forced to pass through the filter and wherein the infection control bedding product also includes a means of releasably sealing the cover.

Optionally, the mattress core is enclosed in a cover which has excess material to provide an overhang of material.

Advantageously, the lip or opening of the cover may be provided with one or more stiffening reinforcement means such as reinforcing batons.

The reinforcing batons may be on both sides, or just at the top, or just at the bottom.

The reinforcing batons are of rectangular, circular, oval or other shape in cross-section, and the reinforcing batons may be substantially flexible or substantially rigid.

In one aspect, the present invention provides a valve having associated means for detecting presence of microbes in the air flowing through the valve.

In one embodiment, the valve includes the detection means, the detection means being visible to the human eye.

Advantageously, the microbial detection means may detect and indicate the presence of microbes by including a detection means which directly indicates the presence of microbes in air passing through the valve or within the hermetically sealed infection control cushioning and bedding device.

In an alternative embodiment, the microbial detection means associated with the valve comprises a two-part indicator means whereby:
a first component microbial detection means such as a tablet composed of certain materials is included within the hermetically sealed infection control cushioning and/or bedding device; as a consequence of bacteriological or other microbial breakdown, an activator compound is released from the first component microbial detection means; the valve, including a second component microbial detection means such as an indicator substance which is specific to the activator compound included in the first compound and the indicator substance detects the presence of the activator which, in turn, indicates microbial presence within the hermetically sealed infection control cushioning and/or bedding device.

The present invention also provides hermetically sealed infection control cushioning and/or bedding devices such as mattresses, cushions, pillows, duvets, or such like having a sealed cover and having a resiliently deformable filing material comprising a filter medium including a filter membrane for the removal of particles of microbial size whereby the filter medium allows air and water vapour to flow through but is substantially impermeable to liquid while providing a barrier to microbial sized particles and wherein the infection control cushioning and/or bedding devices also include at least one uni-directional valve which has microbial detection means associated with said valve.

Optionally, in one embodiment, the uni-directional valve includes the microbial detection means.

Thus, the present invention relates to the inclusion of one or more microbial detection means into a valve for attachment to the cover of an infection control cushioning and/or bedding device e.g. a pillow and/or mattress cover and such like.

Preferably, the valve is a uni-directional (one-way) valve.

Preferably, the microbial detection means includes at least one of the following means: a pH-sensitive colourant for indicating presence of microbes; metal complexes and solvatochromatic colourants.

U.S. Pat. No. 7,178,555 (Plitek Inc.) discloses a one-way pressure relief valve comprising several die-cut components arranged so as to allow transport of air in one direction only. Such pressure relief valves are typically used for venting gases from bags of coffee.

U.S. Pat. No. 7,178,555 discloses that one embodiment includes a pressure relief valve having a base which may include an adhesive layer and a release liner. The adhesive layer allows the base to be mounted to a package or support surface.

An aperture is included on the base which is often located over an opening on the package. The base may be made of a number of materials known to those of skill in the art including Polyethylene Terephthalate by DuPont Teijin Films.

As is also shown in FIGS. 1-5 of U.S. Pat. No. 7,178,555, inner rails 22 and 24 are provided on base 11. Inner rails 22 and 24 may be an adhesive layer that has been applied to the base or of some other suitable material known to those of skill in the art.

U.S. Pat. No. 7,178,555 also discloses that a film or membrane 50 or 150 is also provided which is supported by inner rail 135 or inner rails 22 and 24. The film may be made of Polyethylene Terephthalate by DuPont.

In operation, as shown in FIG. 3 of U.S. Pat. No. 7,178,555, the film 50 is in a closed position and extends inwardly to cover aperture 20. In this position, the film 50 acts as a seal which prevents exposure to the outside environment.

According to U.S. Pat. No. 7,18,555, to vent the contents of a container 16, the film 50 moves into an open position through the force created by the pressurized gas located in container 16. The pressure moves film 50 up off of aperture 20. This, in turn, permits aperture 20 to be in communication with the passageway so as to allow venting to occur. Once the pressure in the package equalizes, the film 50, again, moves inwardly to cover and seal off the aperture.

As is also shown in FIG. 3 of U.S. Pat. No. 7,178,555, in an open position, the passageway and aperture form an opening that is stepped in configuration which permits venting to occur. By permitting the film 50 to move downwardly and upwardly within space defined by the passageway, contact by extraneous surfaces or objects with film 50 will not interfere with the operation of valve 50.

Although not disclosed in U.S. Pat. No. 7,178,555, it is known that components of the valve of U.S. Pat. No. 7,178,555 may be formed of transparent materials.

It has been found in practice that correct functioning of these valves requires a carefully metred quantitiy of oil being injected between two layers in the valve, on assembly.

Thus, in practice it was found that by adding a drop of oil to the valve assembly in the recess immediately underneath the film 50 assists in forming a seal and also lubricates and facilitates the movement of the film during pressure release.

Since the oil is inside the film, it only contacts the atmosphere inside the valve when the valve is closed. Since the valve is open only when gas is exhausting through the valve, i.e. there is substantial flow through the valve, the oil on the inside surface of the membrane 50 is never exposed to the atmosphere outside.

In accordance with one embodiment of the present invention, there is provided a uni-directional valve having a microbial detection means associated with the valve, thereby making it possible to identify the presence of pathogens, or metabolic products of pathogens, contained in the air passing through the valve. Any changes in colour of the microbial detection reagent can be visibly seen, on inspection, through the transparent layers (windows) of the uni-directional valve.

Optionally, the uni-directional valve may include the microbial detection means.

In one embodiment of the present invention, the microbial detection means may be a suitable microbial detection reagent and the microbial detection means may be mixed into the oil included in the uni-directional valve.

According to the present invention, various microbial detection means such as the microbial detection reagents described hereinbelow are miscible with various types of oil, or liquids acting as the oil for this purpose. Examples include mineral oil, silicone oil, glycerine, or there substances miscible with oil-soluble or polar-soluble dyes as necessary.

In an alternative embodiment of the present invention, an additional membrane can be included in the valve assembly, at any layer inside the exhaust membrane component. The additional membrane may comprise a highly porous membrane, eg a woven or non woven or air blown membrane, impregnated or partially impregnated with a suitable microbial detection means such as a microbial detection reagent.

Various suitable methods and substances are available to detect and disclose the presence of pathogens.

One method of detecting the presence of pathogens is by monitoring changes in pH resulting from bacterial activity.

In accordance with the present invention, in one embodiment, the oil within the uni-directional valve may comprise a pH-sensitive colourant capable of changing colour in the presence of one or more microorganisms. That is, the colourant may change from a first colour to a second colour or from colourless to a colour or from a colour to colourless. PH-sensitive indicators exist that are capable of differentiating between certain types of microorganisms. Bacteria, for example, may metabolize the growth medium and generate acidic compounds (e.g., CO2) or alkaline compounds (e.g., ammonia) that lead to a change in pH. Likewise, certain microorganisms (e.g., bacteria) contain highly organised acid moieties on their cell walls. Because the acidic/basic shift may vary for different microorganisms, pH-sensitive colourants can be selected as appropriate for the desired pH transition so as to detect the presence of specific organisms.

Examples of such pH-sensitive colourants include: Phthalein colourants, many of which may be selected to give specific changes at specific pH values.

In an alternative embodiment of the invention, metal complexing can be used to detect and indicate the presence of microbes. For example, many microorganisms (e.g., bacteria and fungi) produce low molecular weight iron-complexing compounds in growth media, which are known as “siderophores.” Metal complexing indicators may undergo a colour change in the presence of siderophores. Preferred metal complexing indicators include aromatic azo compounds, eg Eriochrome Black T, Eriochrome Blue SE, Eriochrome Blue Black B, Eriochrome Cyanine R, Xylenol Orange and many others.

In a further alternative embodiment of the invention, solvatochromatic colourants can be used to detect and indicate the presence of microbes. Solvatochromatic colourants change colour in the presence of a broad range of microorganisms. The colour change occurs as a result of changes in the polar environment. For example, a solvatochromatic colourant may be blue in a polar environment (e.g., water), but yellow or red in a non-polar environment (e.g., lipid-rich solution). Examples of suitable colourants are Merocyanine colourants, zwitterionic colourants (eg N-phenolate betaine colourants), or Reichardt's dye; 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenox-azin-5-one (Nile Red); 4-(dicyanovinyl)julolidine (DCVJ); phenol blue; and many others, or mixtures thereof

It should be understood that the present invention is not limited to any particular mechanism for the colour change. Even when a pH-sensitive colourant is employed, for instance, other mechanisms may actually be wholly or partially responsible for the colour change of the colourant. For example, redox reactions between the colourant and microorganism may contribute to the colour change.

In one embodiment of the present invention, the microbial detection means such as one of the colourants described above, is impregnated into the valve by mixing the microbial detection means with the oil referred to above, inside the valve. The valve can be in the form of a single self-adhesive valve for attachment to a mattress cover. In use, air is periodically expelled from the mattress, and all or some of this air must pass through the valve. The air, having been static within the mattress for some time, will be rich in any microbial metabolites, if any are present. If these are present in sufficiently large quantities, the colourant contained within the valve will change colour.

This colour change may be visible to the naked eye, or visible when illuminated by fluoroscopic or other methods.

The dye may be chosen to be sensitive to microbial activity in general, or specific categories of organism such as gram positive or gram negative bacteria.

In a further embodiment, two or more valves may be provided, each valve adapted to be sensitive to a different category of organism. In this way, a profile of the colonisation in the bedding may be visible. With a greater number of valves in the array, a more detailed profile can be generated.

In a further aspect of the present invention, the present invention also provides a hermetically sealed infection control bedding product such as a mattress, cushion, pillow, duvet, or such like having a sealed cover and having a resiliently deformable filling material comprising a filter medium including a filter membrane for the removal of particles of microbial size whereby the filter medium allows air and water vapour flow through but is substantially impermeable to liquid while providing a barrier to microbial sized particles and wherein the infection control bedding product also includes a uni-directional valve to allow air to be vented from the pillow whereby any air entering the pillow is forced to pass through the filter.

To prevent cross-infection, it is only necessary to provide microbial filtering of the air in one direction. This is because, since only sterile air ever enters the pillow, no microbes can incubate there and consequently any air exhausting from the pillow is sterile and so need not be filtered.

The present invention accordingly has the advantage that it provides a one-way valve incorporated into the pillow cover as well as the microbial filter membrane, to allow air to exhaust quickly from the pillow through the valve, bypassing the membrane. Air re-entering the pillow may only pass through the microbial filter membrane. Thus, air re-entering the pillow does so at slower speed, but has the advantage that forcing the re-entering air to pass through the micorbial filter allows only sterile air to enter the pillow.

The speed of return of air into the pillow following use is not a problem. When changing head position, for example, the pillow is quickly re-loaded, and so approximately the same volume of air is required before and after movement. After use, of course, it does not matter how quickly the pillow re-inflates, over a period of minutes.

In one embodiment, the uni-direction valve (one way valve) may be in the form of an inexpensive self-adhesive die-cut construction which is typically found on coffee bags (such as that disclosed in U.S. Pat. No. 7,178,555 (Plitek Inc)), or alternatively, may be in another form of uni-directional valve. There are numerous suitable designs. Preferably, the one-way valve is attached to the hygienic bedding item by punch-welding, adhesive or other suitable process.

The present invention also provides hermetically sealed infection control cushioning and/or bedding devices such as mattresses, cushions, pillows, duvets, or such like having a sealed cover and having a resiliently deformable filing material comprising a filter medium including a filter membrane for the removal of particles of microbial size whereby the filter medium allows air and water vapour to flow through but is substantially impermeable to liquid while providing a barrier to microbial sized particles and wherein the infection control cushioning and/or bedding devices also include at least one uni-directional valve which has microbial detection means associated with said valve.

In one embodiment, the microbial detection means associated with the valve comprises a two-part indicator means whereby:
a first component microbial detection means such as a tablet composed of certain materials is included within the hermetically sealed infection control cushioning and/or bedding device; as a consequence of bacteriological or other microbial breakdown, an activator compound is released from the first component microbial detection means; the valve, including a second component microbial detection means such as an indicator substance which is specific to the activator compound included in the first compound and the indicator substance detects the presence of the activator which, in turn, indicates microbial presence within the hermetically sealed infection control cushioning and/or bedding device.

In this particular embodiment, the microbial detection means associated with the valve comprises a two-part indicator means wherein the first component microbial detection means is in the form of:
a tablet composed of certain materials and this tablet is contained within the pillow or bedding; as a consequence of bacteriological or other microbial breakdown, a ‘chemical compound X’ is released; the valve contains an indicator substance which is specific to ‘chemical compound X’; and the indicator substance detects the present of chemical compound X which, in turn, indicates microbial presence within the pillow or bedding.
   Advantages of this System of Detection are:
1. False positives are minimised, because the indicator is only sensitive to compound X, and compound X is only released within the pillow as a result of degradation of the tablet. Thus, the indicator is not sensitive to contaminants from the outside of the valve.
2. Whilst the indicator may be very specific to compound X, the conditions leading to the degradation of the tablet may be quite general if so desired. For example, degradation due simply to excessive moisture, or due to the action of a broad range of organisms, may lead to the breakdown of the tablet and so the release of compound X.
3. The system allows for the setting of a threshold level of biological activity, below which no indication will be made. For example, compound X may be contained within an inner layer of the tablet, and is released only when the outer layer is breeched by degradation.

Numerous pairs of substances may be used as the first component detection means including an activator material and second component detection means (an indicator), and many examples of such substances are described elsewhere. For example, a colloidal suspension of amylose (e.g. impregnated into a porous medium within the valve) would turn deep blue when exposed to iodine. Iodine is a solid, which sublimes in small quantities at room temperature. Thus, encapsulated in a biodegradable tablet, degradation of outer shell of the tablet would result in the evolution of small quantities of iodine gas. This gas, exhausting through the valve, would irreversibly activate the amylose indicator.

Many other pairs of such pairs of substances may be chosen to work in a similar way.

Examples using non-hazardous substances which can function as the first component detection means (activator compound) and second component detection means (indicators) include:

Benedict's solution (salts of sodium and copper), turning green in the presence of sugar. (Sugar released/dissolved/transported on breakdown of tablet).

Buiret solution (turning purple in the presence of certain proteins).

Sudan III, turning red in the presence of fat, eg vegetable oil.

Dichlorophenolindophenol, turning from blue to clear in the presence of vitamin C. (We could even use an effervescent vitamin C tablet as our compund X tablet).

Plus many many more!

### Embodiments/Variants:

1. Tablet of dry substance X contained within pillowcase. Indicator Y in valve. Excessive moisture/degradation causes solution and transport, or gas evolution of X.
2. Substance X is encapsulated within a tablet. One example may be a sugar-coating, as in Nurofen tablets. X is released when coating is breeched by degradation.
3. Substance X is distributed as a talc throughout the interior of the pillow. Moisture anywhere in the pillow causes solution, transport, or gas evolution of substance X.
4. Substance x is applied by spray or other means as a coating to the inside of the cover fabric.
5. Substance X may be preferentially located adjacent to the indicator valve.
6. Substance X may be preferentially located as an additional layer within the indicator valve, so that both of the stages of the 2-stage indication process take place within the valve.

The indicator may consist of a colour-changing material e.g. a leuco dye combined with a developing agent, such that the developing agent is specific to substance X, and produces a pH change to activate the leuco dye. Developing agents may be selected from phenolic resins or phenolic compounds such as 4-tert-butylphenol; 4-phenylphenol; methylene-bis(p-phenylphenol); 4-hydroxydiphenyl ether; alpha-naphthol; beta-napthol; methyl 4-hydroxybenzoate; benzyl 4-hydroxybenzoate; 4-hydroxydiphenyl sulfone; 4-hydroxyacetophenone; 2,2′-dihydroxydiphenyl; 4,4′-cyclohexylidenephenol; 4,4′-isopropylidenediphenol; 4,4-isopropylidenebis(2-methylphenol); a pyridine complex of zinc thiocyanate; 4,4-bis(4-hydroxyphenyl)valeric acid; hydroquinone; pyrogallol; phoroglucine; p-hydroxybenzoic acid; m-hydroxybenzoic acid; o-hydroxybenzoic acid; gallic acid; 1-hydroxy-2-naphthoic acid.

One aim of the present invention is to provide a means of determining the contamination status of the interior of infection control cushioning and/or bedding devices including particularly, but not exclusively, cushioning and/or bedding devices such as mattresses, pillows, cushions, duvets and other sealed cushioning and bedding devices without compromising the functionality of the cushioning and bedding devices which include microbial filter media for filtering microbes out of the air entering the cushioning and bedding devices.

In one aspect of the invention, the present invention provides a hermetically sealed infection control bedding product such as a mattress, cushion, pillow, duvet, or such like having a sealed cover and having a resiliently deformable filling material comprising a filter medium in the cover, wherein the filter medium comprises a filter membrane for the removal of particles of microbial size whereby the filter medium allows air and water vapour flow through but is substantial impermeable to liquid while providing a barrier to microbial sized particles and wherein the infection control bedding product also includes a uni-directional valve to allow air to be vented from the pillow whereby any air entering the pillow is forced to pass through the filter and wherein the infection control bedding product also includes a means of releasably sealing the cover.

The hygienic bedding product in accordance with the invention will now be described more particularly with reference to the accompanying drawings, in which are shown a number of embodiments of the hygienic bedding product in one aspect of the present invention.

### IN THE DRAWINGS

FIG. 1 is a plan view of a first embodiment of a pillow with a waterproof cover including a waterproof microbial filter membrane in a first area of the pillow and a unidirectional venting valve in a second area of the pillow, remote from the first area;

FIG. 2 is a perspective view of a mattress with a waterproof covering and a unidirectional valve and microbial filter (not shown) and a means of sealing the end of the cover while enabling visual inspection of the mattress;

FIG. 3 is a sectional side view of the mattress embodiment of FIG. 2;

FIG. 4 is a plan view of the mattress shown in FIGS. 2 and 3;

FIG. 5 is a plan view of the mattress of FIG. 4 but now showing a sealable cover for the rolled-up portion of the waterproof covering of the mattress; and

FIG. 6 is a perspective view of the mattress shown in FIG. 5.

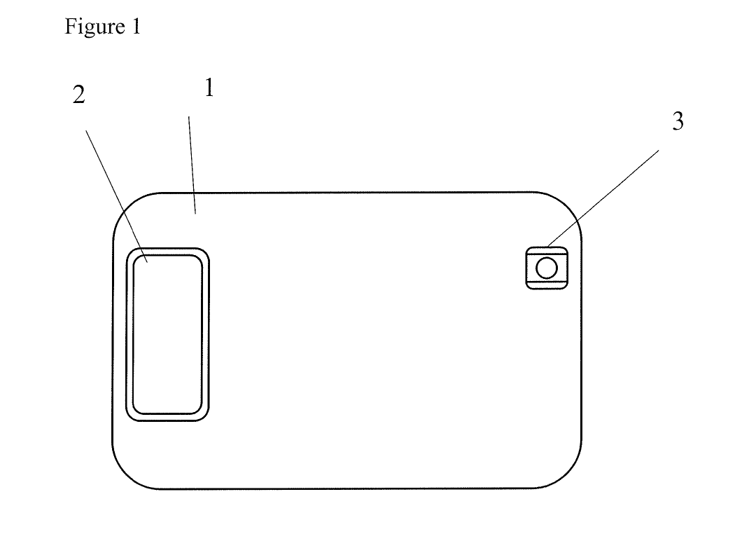

Referring initially to FIG. 1, one preferred embodiment of the present invention comprises of a pillow with a waterproof cover, 1, wherein one part of the cover includes an area comprising a waterproof microbial filter membrane, 2, and elsewhere on the cover is attached a unidirectional exhaust valve, 3.

In an alternative embodiment, the valve may be incorporated into the filter area of the cover in the area comprising the waterproof microbial filter membrane.

It is to be understood that a pillow is described and shown here by way of example only and that the same approach may also be adopted with sealed duvets, and mattresses and cushions.

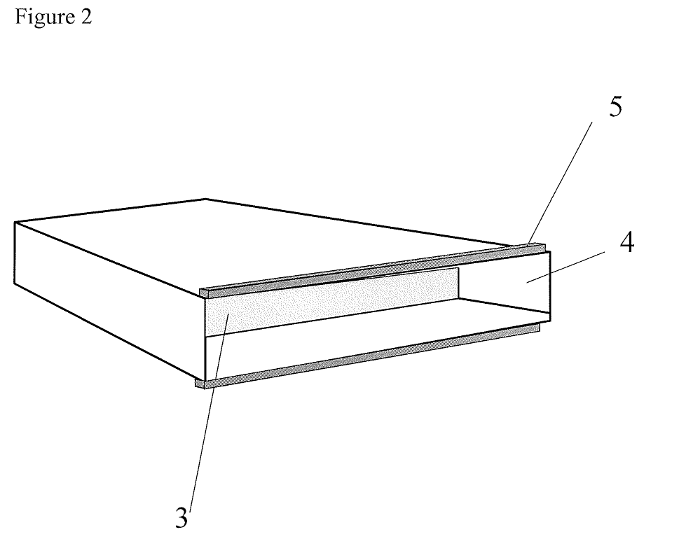

Referring to FIGS. 2, 3 and 4, which show an alternative aspect of the invention, a means for sealing the mattress in a waterproof covering for enabling visual inspection will be described.

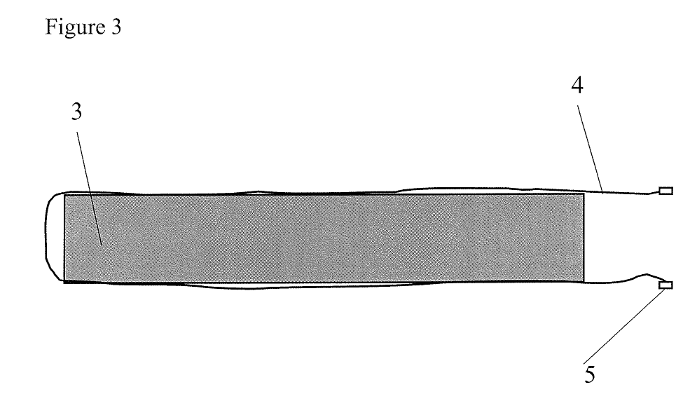

Referring now to FIG. 2, the mattress core, 3, is enclosed in a cover, 4, which has excess material to provide overhang. FIG. 3 shows a sectional side view of the same assembly. The lip or opening of the cover may be provided with one or more stiffening reinforcement such as reinforcing batons 5. These may be on both sides, or just at the top, or just at the bottom. The stiffening reinforcements, 5, may be of rectangular, circular, oval or other shape in cross-section, and the stiffening reinforcements 5 may be substantially flexible or substantially rigid.

This arrangement has the advantage that it provides a waterproof seal while enabling inspection of the mattress inside the cover.

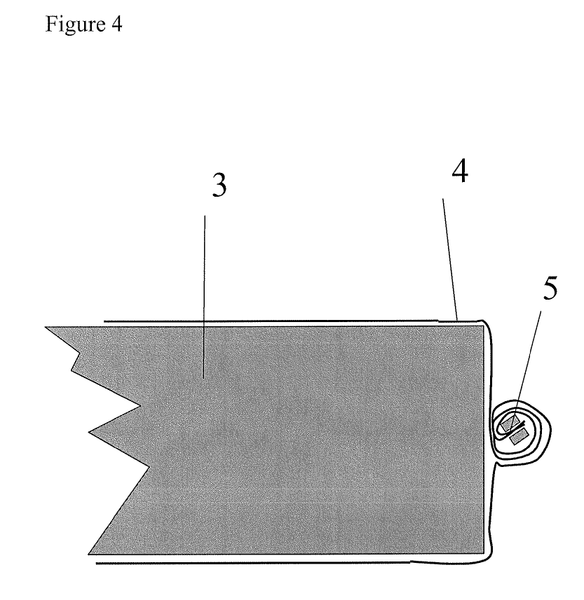

Referring to FIG. 4, the stiffening reinforcements 5 may be used to roll up the excess cover to form a seal.

In an alternative embodiment, the end of the cover may be rolled up without any reinforcement means.

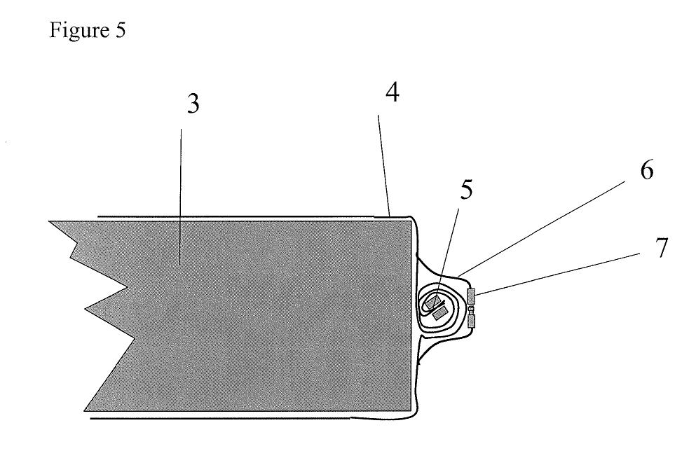

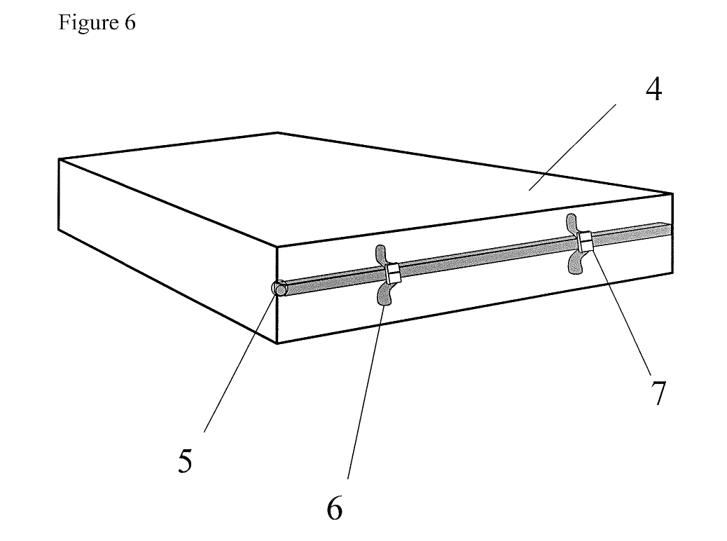

FIG. 5 and FIG. 6 show the cover in its final, tightly-rolled position fixed with one or more securing means such as straps, 6, and buckles, 7. Other means of fastening may be used such as zips, poppers, hook and loop fastening, toggles, buttons tie-cord, etc.

It is to be understood that the invention is not limited to the specific details described herein which are given by way of example only and that various modifications and alterations are possible without departing from the scope of the invention as defined in the appended claims.

1. A valve for attachment to a cover of an infection control cushioning and/or bedding device, the valve including
means for detecting presence of microbes in air flowing through the valve, thereby making it possible to identify the presence of pathogens, or metabolic products of pathogens, contained in the air passing through the valve.

2. A valve as claimed in claim 1, wherein the valve is uni-directional and only allows air to flow outwardly from the infection control cushioning and/or bedding device in which the valve is included.

3. A valve as claimed in claim 1, wherein the microbial detection means includes at least one of the following group: a microbial detection reagent; a pH-sensitive colourant for indicating presence of microbes; metal complexes and solvatochromatic colourants.

4. A valve as claimed in claim 1, wherein the valve comprises oil and the microbial detection means is included in the oil.

5. A hermetically sealed infection control cushioning and/or bedding device having a sealed cover and a resiliently deformable filing material, the hermetically sealed infection control cushioning and/or bedding device comprising
a filter medium including a filter membrane for the removal of particles of microbial size whereby the filter medium allows air and water vapour to flow through but is substantially impermeable to liquid while providing a barrier to microbial sized particles and
at least one valve which has microbial detection means associated with said valve.

6. A hermetically sealed infection control cushioning and/or bedding device as claimed in claim 5, wherein the valve is uni-directional and only allows air to flow outwardly from the hermetically sealed infection control cushioning and/or bedding device.

7. A hermetically sealed infection control cushioning and/or bedding device as claimed in claim 5, wherein the valve includes the microbial detection means.

8. A hermetically sealed infection control cushioning and/or bedding device as claimed in claim 5, wherein the microbial detection means includes at least one of the following group: a microbial detection reagent; a pH-sensitive colourant for indicating presence of microbes; metal complexes and solvatochromatic colourants.

9. A hermetically sealed infection control cushioning and/or bedding device as claimed in claim 5, wherein the valve comprises oil and the microbial detection means is included in the oil.

10. A hermetically sealed infection control cushioning and/or bedding device as claimed in claim 5, wherein the microbial detection means associated with the valve comprises a two-part detection means wherein:
a first component of the detection means is contained within the hermetically sealed infection control cushioning and/or bedding device;
the first component comprises an activator material which is releasable or degradable as a consequence of degradation
the valve includes a second component of the detection means, the second component comprising an indicator which is specific to detecting the activator material included in the first component of the detection means, whereby the indicator detects the presence of the activator material released from the first component of the detection means which, in turn, indicates microbial presence within the hermetically sealed infection control cushioning and/or bedding device.

11. A hermetically sealed infection control cushioning and/or bedding device as claimed in claim 10, wherein the activator material is contained within a degradable carrier in the first component of the detection means, whereby degradation of the carrier results in release of the activator material.

12. A hermetically sealed infection control cushioning and/or bedding device as claimed in claim 10, wherein the degradation is due to bacteriological or other microbial presence in the hermetically sealed infection control cushioning and/or bedding device.

13. A hermetically sealed infection control cushioning and/or bedding device having a sealed cover and a resiliently deformable filling material, the hermetically sealed infection control cushioning and/or bedding device comprising
a filter medium including a filter membrane for the removal of particles of microbial size whereby the filter medium allows air and water vapour flow through but is substantially impermeable to liquid while providing a barrier to microbial sized particles; and
a uni-directional valve to allow air to be vented from the pillow whereby any air entering the pillow is forced to pass through the filter.

14. A hermetically sealed infection control cushioning and/or bedding device as claimed in claim 13 having a waterproof cover, wherein one part of the cover includes an area comprising a waterproof microbial filter membrane, and elsewhere on the cover is attached a unidirectional exhaust valve.

15. A hermetically sealed infection control cushioning and/or bedding device having a sealed cover and a resiliently deformable filling material, the hermetically sealed infection control cushioning and/or bedding device comprising
a filter medium in the cover, wherein the filter medium comprises a filter membrane for the removal of particles of microbial size whereby the filter medium allows air and water vapour flow through but is substantial impermeable to liquid while providing a barrier to microbial sized particles;
a uni-directional valve to allow air to be vented from the pillow whereby any air entering the pillow is forced to pass through the filter; and
means of releasably sealing the cover.

16. A hermetically sealed infection control cushioning and/or bedding device as claimed in claim 15, wherein a mattress core is enclosed in the cover which has excess material to provide an overhang of material.

17. A hermetically sealed infection control cushioning and/or bedding device as claimed in claim 16, wherein a lip or opening of the cover may be provided with one or more stiffening reinforcement means.

18. A hermetically sealed infection control cushioning and/or bedding device as claimed in claim 17, wherein the reinforcement means include at least one reinforcing baton.

19. A hermetically sealed infection control cushioning and/or bedding device as claimed in claim 18, wherein the at least one reinforcing baton is of rectangular, circular, oval or other shape in cross-section.

20. (canceled)
